(19) United States
(12) Patent Application Publication
(10) Pub. No.: US 2024/0059635 A1
(43) Pub. Date: Feb. 22, 2024

(54) PROCESS FOR THE PRODUCTION OF 1,1-DIFLUOROETHANE

(71) Applicant: Mexichem Fluor S.A. de C.V., San Luis Potosi (MX)

(72) Inventors: Andrew SHARRATT, Runcorn (GB); Claire REES, Runcorn (GB)

(21) Appl. No.: 18/259,324

(22) PCT Filed: Jan. 19, 2022
(86) PCT No.: PCT/GB2022/050145

§ 371 Date: Jun. 26, 2023

(30) Foreign Application Priority Data
Jan. 22, 2021 (GB) 2100874.3

(51) Int. Cl. C07C 17/087; C07C 17/383; C07C 19/08; B01J 23/26; B01J 23/06

(52) U.S. Cl. CPC C07C 17/087; C07C 17/383; C07C 19/08; B01J 23/26; B01J 23/06

(57) ABSTRACT

A process for the production of 1,1-difluoroethane by the catalytic fluorination, in the vapour phase, of a composition comprising vinyl chloride with hydrogen fluoride, wherein the vinyl chloride is contacted with hydrogen fluoride, at temperatures between 100 and 500° C., in the presence of a catalyst comprising a one or more of chromia, alumina, carbon.

The present invention is concerned with the preparation of 1,1-difluoroethane (HFC-152a). More particularly, the present invention is concerned with a process for the preparation of 1,1-difluoroethane (HFC-152a) comprising fluorinating vinyl chloride (H₂C═CHCl, R-1140).

Hereinafter, unless otherwise stated, 1,1-difluoroethane will be referred to as HFC-152a. HFC-152a is known to have utility as, for example, an aerosol propellant, a foam expansion agent, and as a refrigerant. HFC-152a has zero Ozone Depletion Potential (ODP) and very low Global Warming Potential (GWP).

There are methods known in the art for producing HFC-152a.

CN1931431A describes how 1,1-difluoroethane is obtained by passing a mixture of vinyl chloride and hydrogen fluoride over a chromium oxyfluoride catalyst, including an additional metal selected from cobalt, manganese, zinc, iron, magnesium, aluminium, or nickel in the gas phase.

US2005222472 describes the use of an activated carbon catalyst impregnated with a Lewis acid, to convert to HFC152a in the vapour phase.

GB921254A describes how vinyl fluoride and 1,1-difluoroethane are obtained by passing a mixture of ethylene dichloride or vinyl chloride or both and hydrogen fluoride over a chromium oxide catalyst in the vapour phase. Vinyl chloride and 1,1-difluoroethane may be separated from the reaction product and recycled.

RU2614442 describes a method for obtaining 1,1-difluoroethane, consisting of liquid-phase fluorination of vinyl chloride in the presence of hydrogen fluoride, catalysed by tin tetrachloride, followed by removal of remaining vinyl chloride in a vapour-phase reaction with hydrogen fluoride over an aluminium oxide-based supported metal catalyst.

However, these methods suffer from a range of disadvantages such as low yields, and/or the handling of toxic and/or expensive reagents, and/or the use of extreme conditions, and/or the production of toxic by-products, but especially high rates of catalyst deactivation caused by coke generation.

Excessive coke generation is wasteful from the perspective of poor selectivity/conversion of the starting material. Additionally, coke generation is known to reduce catalyst efficacy, deactivating a catalyst by blocking active sites, meaning that production of the desired product is impeded and (excessive) time is needed for catalyst regeneration to remove the undesired coke deposits.

There is therefore a need for a more economically efficient means for producing HFC-152a. In particular, there is a need to provide a more efficient manufacturing process in which the selectivity towards HFC-152a and the yield thereof is sufficiently high and in which catalyst deactivation by coke formation is minimised, so that the HFC-152a may be produced, sold or used in an economically valuable way.

The listing or discussion of a prior-published document in this specification should not necessarily be taken as an acknowledgement that the document is part of the state of the art or is common general knowledge.

According to a first aspect of the invention there is provided a process for the production of 1,1-difluoroethane (HFC-152a) by the catalytic fluorination, in the vapour phase, of a composition comprising vinyl chloride with hydrogen fluoride, wherein the vinyl chloride is contacted with hydrogen fluoride, at temperatures between 100 and 500° C., in the presence of a catalyst comprising one or more of chromia, alumina, carbon.

The process has been found to produce HFC-152a in high yield with a high selectivity to HFC-152a accompanied by low rates of coke formation and loss of catalyst activity.

Generally the yield of HFC-152a has been found to be high with preferably over 80 wt % of vinyl chloride being converted in the reaction. More preferably over 85 wt %, more preferably over 87 wt %, more preferably over 90 wt %, more preferably over 92 wt %, more preferably over 95 wt %, more preferably over 97 wt %, more preferably over 98 wt %, more preferably over 99 wt %.

Generally the selectivity to HFC-152a has been found to be high with preferably over 80 wt % of vinyl chloride being converted to HFC-152a in the reaction. More preferably over 85 wt %, more preferably over 87 wt %, more preferably over 90 wt %, more preferably over 92 wt %, more preferably over 95 wt %, more preferably over 97 wt %, more preferably over 98 wt %, more preferably over 99 wt %.

In an alternative/additional way of expressing the selectivity of the reaction it has been found that the weight percentage of by-products in the reactions (such as HFC-151a and/or HFC-150a is less than 15 wt %, more preferably less than 13 wt %, more preferably less than 10 wt %, more preferably less than 8 wt %, more preferably less than 5 wt %, more preferably less than 3 wt %, more preferably less than 2 wt %, more preferably less than 1 wt %.

Thus the composition of the product of the reaction comprises less than 15 wt %, more preferably less than 13 wt %, more preferably less than 10 wt %, more preferably less than 8 wt %, more preferably less than 5 wt %, more preferably less than 3 wt %, more preferably less than 2 wt %, more preferably less than 1 wt % of HFC-151a and/or HFC-150a.

Prior to the fluorination, the catalyst is usually subjected to an activation treatment to achieve the desired catalytic performance. Normally, this involves treating the catalyst with hydrogen fluoride, at an elevated temperature and pressure. Frequently, the activation treatment is preceded by other steps, such as drying or heating the catalyst under an inert atmosphere.

The catalyst comprises one or more of chromia, alumina and/or carbon.

Preferably the catalyst comprises at least one additional metal or compound thereof, wherein at least one additional metal is selected from Li, Na, K, Ca, Mg, Cs, Sc, Al, Y, Ti, Zr, Hf, V, Nb, Ta, Cr, Mo, W, Mn, Re, Fe, Ru, Co, Rh, Ir, Ni, Pd, In, Pt, Cu, Ag, Au, Zn, La, Ce and mixtures thereof. Most preferably the additional metal is zinc.

This additional metal or compound thereof can also be referred to as a promoter.

Preferably, the catalyst is provided in the form of a pellet or pellets comprising a plurality of catalyst particles. Such catalyst particles may be pressed together, for example under load, to form the pellets. The pellets may comprise one or more further materials. For example, the pellets may include graphite, preferably in an amount of from about 0.5 wt % to about 10 wt %, e.g. from about 1 wt % to about 5 wt %. Preferably, the pellets have a longest dimension from about 1 mm to about 100 mm. In some embodiments, the pellets may have a longest dimension of about 1 mm to about 10 mm, for example from about 3 mm to about 5 mm.

Preferably, the catalyst comprises at least 80 wt % (for example at least 85 wt %, at least 90 wt %, at least 92 wt %, at least 93 wt %, at least 94 wt %, at least 95 wt % or at least 96 wt %) chromia. Advantageously, the catalyst may be a zinc/chromia catalyst. By the term “zinc/chromia catalyst” we mean that the metal oxide catalyst comprises chromium or a compound of chromium and zinc or a compound of zinc.

The total amount of the zinc or a compound of zinc present in the zinc/chromia catalysts of the invention is typically from about 0.01% to about 25%, preferably 0.1% to about 25%, conveniently 0.01% to 6% of the catalyst; and in some embodiments preferably 0.5% by weight to about 25% by weight of the catalyst, preferably from about 1 to 10% by weight of the catalyst, more preferably from about 2 to 8% by weight of the catalyst, for example about 3 to 6% by weight of the catalyst.

Additional metals or compounds thereof are typically present from about 0.01% to about 25%, preferably 0.1% to about 25%, conveniently 0.01% to 6% by weight of the catalyst; and in some embodiments preferably 0.5% by weight to about 25% by weight of the catalyst, preferably from about 1 to 10% by weight of the catalyst, more preferably from about 2 to 8% by weight of the catalyst, for example about 3 to 6% by weight of the catalyst.

In other embodiments, the catalyst may be an alumina catalyst with one or more promoters selected from platinum, iron, chromium and zinc. The total amount of promoter is typically from about 0.1 to about 60% by weight of the catalyst, preferably from about 0.5 to about 50% by weight of the catalyst, such as 0.5% by weight to about 25% by weight of the catalyst, or from about 1 to 10% by weight of the catalyst. In such embodiments it is preferred that the catalyst comprises at least 80 wt % (for example at least 85 wt %, at least 90 wt %, at least 92 wt %, at least 93 wt %, at least 94 wt %, at least 95 wt % or at least 96 wt %) alumina. In some embodiments, the catalyst may be in fluorinated form. For example, the catalyst may have been fluorinated by treatment with HF at elevated temperature.

The process is conducted in the vapour phase. The process may be carried out at atmospheric, sub- or super atmospheric pressure, typically at from 0 to about 30 barg, preferably from about 1 to about 20 barg, preferably from about 5 to about 15 barg, such as about 10 barg.

The process typically employs a molar ratio of vinyl chloride with hydrogen fluoride of from 1:1 to 1:50, more preferably 1:1 to 1:25, 1:1 to 1:15, 1:1 to 1:10 or in a range of from 1:5-30, such as 1:10-25.

The reaction time for the process generally is from about 1 second to about 1000 hours, preferably from about from about 1 second to about 200 hours, more preferably from about 10 seconds to about 50 hours, such as from about 1 minute to about 10 or 20 hours. In a continuous process, typical contact times of the catalyst with the reagents are from about 1 to about 1000 seconds, such from about 1 to about 500 seconds or about 1 to about 300 seconds or about 1 to about 50, 100 or 200 seconds.

The reaction temperature is preferably between 100 and 500° C., more preferably 100 and 300° C., more preferably between 125 and 275° C., more preferably between 150 and 250° C. and most preferably from 175 to 240° C.

The invention will now be illustrated with reference to the following examples.

### EXAMPLES

Vapour Phase Reaction of Vinyl Chloride with HF

The following steps were followed.

### Catalyst Activation

The reactor bed (4×1.27 cm (½″) outside diameter (OD)×31 cm Inconel 625 reactor tubes connected in series) was charged with catalyst (12 g, 13.29 mL, 6.5% ZnO/Cr₂O₃, 2.00-3.35 mm) supported by Inconel mesh.
The reactors were heated to 250° C. under 80 ml/min nitrogen flow (3 barg) for 16 hours to dry the catalysts.
The catalysts were pre-fluorinated in two stages:—
   Stage 1—3 Barg. 80 ml/min N₂. 4 ml/min HF @ 250° C. After HF was detected in the reactor off-gas, heat to 300° C. and leave overnight.
   Stage 2-3 barg. Reduction of N₂ from 80 ml/min to 40 to 20 to 10 to 5 and then 0 ml/min, over 0.5-1 h per step, allowing the HF flow to stabilise (as measured by titration with 0.1M NaOH_{(aq)}.
The reactors were heated to 380° C. at a rate of 25° C./hour and held at 380° C. for 7 hours before being allowed to cool to the desired reaction temperature, with HF flow on.

### Reaction

HF (125 ml/min) and vinyl chloride (5 ml/min) were passed over the catalyst bed at between 175° C. and 225° C. for a cycle time of about 200 h.
Reactor “off gas” samples were taken and scrubbed through deionised water prior to analysis.
The samples were analysed by gas chromatography (GC) calibrated for vinyl chloride, HFC-152a (1,1-Difluoroethane), HFCC-151a (1-Chloro-1-fluoroethane) and HCC-150a (1,1-Dichloroethane).
At the end of the cycles the HF and vinyl chloride feeds were turned off and the amount of Carbon deposited on the catalysts was determined by performing a regeneration as follows:
   Nitrogen (80 ml/min) and Air (20 ml/min) were passed over the reactors at 225° C., the reactors were then heated at 100° C./hr to 380° C. and the temperature held for 12 h before being cooled to 225° C.
   The “reactor off gases” during regeneration were analysed using a Cambridge Sensotec Rapidox 3100 (a device, that measures O₂, CO₂ and CO in real time as the coke is burned off the catalyst, used to calculate the amount of carbon) to produce a figure for the amount of carbon deposited on the catalyst.

### Example 1

The technique described above was followed (225° C. and 10 barg) for a cycle time of 200 hours.

| | VC | | | | |
|---|---|---|---|---|---|
| | Conversion | Selectivity (%) | | | |
| Run time (h) | (%) | 152a | 151a | 150a | Total C |
| 50 | 99.82 | 99.46 | 0.51 | 0.02 | |
| 100 | 99.83 | 99.55 | 0.53 | 0.01 | |
| 150 | 99.67 | 99.10 | 0.86 | 0.03 | |
| 200 | 99.76 | 99.32 | 0.67 | 0.02 | 1.44% |

The process produces HFC-152a in high yield with a high selectivity, with highly stable catalyst activity.

The production of coke in the reaction is very low (1.44% after 200 hours). Moreover, no loss in conversion or selectivity to 152a is observed with time.

### Example 2

The technique described above was followed (175° C. and 10 barg) for a cycle time of 700 hours.

| | VC | | | | |
|---|---|---|---|---|---|
| | Conversion | Selectivity (%) | | | |
| Run time (h) | (%) | 152a | 151a | 150a | Total C |
| 50 | 99.89 | 99.43 | 0.54 | 0.03 | |
| 100 | 99.88 | 98.74 | 1.16 | 0.08 | |
| 150 | 99.88 | 98.80 | 1.10 | 0.11 | |
| 200 | 99.83 | 98.32 | 1.53 | 0.15 | 0.96% |
| 700 | — | — | — | — | 1.2% |

The process produces HFC-152a in high yield with a high selectivity, with highly stable catalyst activity.

The production of coke in the reaction is very low (0.96% after 200 hours. 1.2% after 700 hours). Moreover, no loss in conversion and only a trivial loss is selectivity to HFC-152a is observed with time.

1. A process for the production of 1,1-difluoroethane (HFC-152a) by catalytic fluorination, in vapour phase, of a composition comprising vinyl chloride with hydrogen fluoride (HE), the method comprising contacting the vinyl chloride with the hydrogen fluoride, at temperatures between 100 and 500° C., in the presence of a catalyst comprising one or more of chromia, alumina, gr carbon.

2. The process according to claim 1, wherein a reaction temperature is between 100 and 300° C.

3. The process according to claim 1, wherein a reaction pressure is between 1 barg and 20 barg.

4. The process according to claim 1, wherein a ratio of vinyl chloride with hydrogen fluoride is from 1:1 to 1:50.

5. The process according to claim 1, wherein the catalyst comprises chromia.

6. The process according to claim 1, wherein the catalyst comprises chromia and zinc.

7. The process according to claim 1, wherein the catalyst comprises at least one additional metal, selected from selected from lithium, sodium, potassium, calcium, magnesium, caesium, scandium, aluminium, yttrium, titanium, zirconium, hafnium, vanadium, niobium, tantalum, chromium, molybdenum, tungsten, manganese, rhenium, iron, ruthenium, cobalt, rhodium, iridium, nickel, palladium, indium, platinum, copper, silver, gold, zinc, lanthanum, cerium and mixtures thereof or a compound thereof.

8. The process according to claim 1, including a purification step, comprising one or more distillation steps, and/F one or more scrubbing trains, and/or one or more phase separation steps.

9. The process according to claim 8, wherein HF is recovered from at least one distillation column and is optionally recycled.

10. The process according to claim 9, wherein the HF is subjected to further purification prior to recycling.

11. A composition comprising 1,1-difluoroethane produced by the process according to claim 1.

12. The process according to claim 1, wherein a ratio of vinyl chloride to hydrogen fluoride is from 1:1 to 1:25, from 1:1 to 1:15, or from 1:1 to 1:10.

13. The process according to claim 1, wherein a ratio of vinyl chloride to hydrogen fluoride is from 1:5 to 1:30, or from 1:10 to 1:25.
